(19) United States
(12) Patent Application Publication
(10) Pub. No.: US 2009/0036938 A1
(43) Pub. Date: Feb. 5, 2009

(54) METHOD AND SYSTEM FOR EXTERNAL COUNTERPULSATION THERAPY

(75) Inventors: Robert Shipley, Woodbury, MN (US); Guy Alvarez, Lino Lakes, MN (US); Rodney W. Salo, Fridley, MN (US); Anand Iyer, Summerville, SC (US); Joseph M. Pastore, Woodbury, MN (US); Joseph Walker, Shoreview, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., ST Paul, MN (US)

(21) Appl. No.: 11/830,508

(22) Filed: Jul. 30, 2007

(51) Int. Cl. A61N 1/04

(52) U.S. Cl. 607/2

(57) ABSTRACT

An improved system for delivering external counterpulsation therapy is described. The system employs muscle stimulation transducers such as cutaneous electrodes in order to stimulate skeletal muscle and/or vascular smooth muscle in synchronization with the cardiac cycle in a manner that increases the fluid pressure within veins and/or arteries during cardiac diastole.

### BACKGROUND

External counterpulsation (ECP) therapy is a non-invasive, outpatient therapy used in the treatment of cardiac disease. As it has been conventionally applied, a set of cuffs is wrapped around the calves, thighs and buttocks of a patient. These cuffs attach to air hoses that connect to valves that inflate and deflate the cuffs. Electrodes are applied to the skin of the chest and connected to an electrocardiograph (ECG) machine. Blood pressure may also be monitored. Inflation and deflation of the cuffs are then electronically synchronized with the heartbeat and blood pressure using the ECG and blood pressure monitors. The ECP treatment compresses the blood vessels in the lower limbs to increase blood flow to the heart. Each wave of pressure is electronically timed to the heartbeat, so that the increased blood flow is delivered to the heart at the time when it is relaxing during diastole. When the heart pumps again during systole, the cuff pressure is released. This lowers resistance in the blood vessels in the legs so that blood may be pumped more easily from the heart. ECP may also encourage the development of collateral blood flow to the heart and thus contribute to the relief of angina symptoms.

### BRIEF DESCRIPTION OF THE DRAWINGS

FIG. 1 illustrates one embodiment of a system for delivering counterpulsation therapy via muscular stimulation.

FIG. 2 illustrates an embodiment of a system for delivering counterpulsation therapy via muscular stimulation that utilizes an implantable cardiac device.

FIG. 3 illustrates the functional components of an exemplary control unit.

FIG. 4 illustrates an exemplary algorithm by the control unit to deliver counter pulsation therapy.

### DETAILED DESCRIPTION

Described herein is an improved system and method for delivering external counterpulsation therapy that involves direct stimulation of skeletal muscle and/or vascular smooth muscle in synchronization with the cardiac cycle in a manner that increases the fluid pressure within veins and/or arteries during cardiac diastole. Contraction of the skeletal muscle compresses the arteries and veins that course through the muscle to increase fluid pressure within the vessels, while contraction of venous and/or arterial smooth muscle causes venous and/or arterial constriction to also increase fluid pressure. As described above, the basic idea of counterpulsation therapy is to increase fluid pressure within blood vessels during cardiac diastole. Stimulation of skeletal and vascular smooth muscle in synchronization with the cardiac cycle efficiently raises the pressure in both arteries and veins during cardiac diastole and, as the skeletal and smooth muscle relaxes, lowers the arterial pressure during the subsequent systolic phase of the cardiac cycle. Periodic application of counterpulsation therapy is useful in the treatment of patients having a number of cardiac conditions. For example, coronary blood flow that supplies the heart is greatest during diastole, and increased aortic pressure during diastole will increase that flow to benefit patients suffering from coronary artery disease. It has also been found that increased aortic pressure brought about by counterpulsation therapy induces the formation of coronary collateral vessels. Increased venous pressure during diastole increases ventricular filling and cardiac output, and the subsequent decrease in arterial pressure during systole decreases the workload of the heart. Both of these effects are beneficial for patients having some degree of heart failure and may help to prevent or reverse deleterious cardiac remodeling.

Counterpulsation therapy via muscular stimulation has a number of advantages over the conventional manner of delivering counterpulsation therapy by external compression of extremities. Contraction of skeletal muscle applies compressive force directly to the vessels that run through the muscle to more efficiently compress both veins and arteries than external compression Muscular stimulation has the further advantage of producing a training effect for the patient that mimics physical exercise. Another advantage is that, unlike conventional external counterpulsation therapy, pressure can be increased in blood vessels located in body regions other than extremities. Although it may be preferable and most convenient to stimulate muscle tissue in the legs or arms, stimulation could also be applied to muscle tissue in the abdomen and buttocks, for example.

An exemplary system is equipped with one or more muscle stimulation transducers and a control unit that actuates the muscle stimulation transducers during the diastolic phase of the cardiac cycle as determined by detecting cardiac electrical activity or by detecting another physiological variable reflective of the cardiac cycle such as blood pressure or heart sounds. A muscle stimulation transducer may be any device that delivers energy to muscular tissue in a manner that causes contraction of the tissue such as a unipolar electrode, bipolar or multi-polar electrode set of electrodes, a radio-frequency transducer, a magnetic transducer, or an ultrasonic transducer. Although such muscle stimulation transducers could be of an implantable type, they are preferably adapted for transcutaneous stimulation of muscle. The muscle stimulation transducers may be adapted for cutaneous placement near the muscular tissue selected for stimulation in a number of different ways. For example, muscle stimulation transducers may be directly affixed to the skin by adhesive or other means. In another embodiment, the muscle stimulation transducers are incorporated into a garment or structure that is worn by the patient such as a cuff, sock, glove or pants that dispose the muscle stimulation electrodes near the muscular tissue to be stimulated. In another embodiment, the muscle stimulation transducers are incorporated into a patient-support structure such as a mat, a chair, or a recliner that dispose the muscular stimulation electrodes near the muscles of an extremity when the patient is supported thereon.

In order to properly time the delivery of muscle stimulation in relation to the cardiac cycle, the control unit either incorporates, or is interfaced to, a cardiac sensor for detecting cardiac activity. Such a cardiac sensor may be, for example, a surface ECG apparatus that generates electrical signals reflective of the depolarization corresponding to cardiac contraction and the electrical repolarization corresponding to cardiac relaxation. Alternatively, the control unit could communicate via wireless telemetry with a cardiac device implanted in the patient having cardiac sensing capability such as a pacemaker or ICD. Such implantable devices generate electrogram signals analogous to surface ECG signals via internally disposed electrodes. Both ECG and electrogram signals reflect the electrical activity of the heart and contain cardiac activity markers such as T waves and R waves indicative of the phases of the cardiac cycle. The objective is to actuate the one or more muscular stimulation transducers during the diastolic phase of the cardiac cycle as determined from the detected cardiac activity. For this purpose, the control unit and/or cardiac sensor may incorporate filtering and other signal processing circuitry for detecting R waves and/or T waves that correspond to the beginning of systole and diastole, respectively. Alternatively, the cardiac sensor may detect cardiac activity from measurements or detection of physiological variables reflective of the mechanical activity of the heart such as blood pressure, heart sounds, or blood flow. The control unit could also employ a combination of different types of cardiac sensors as described above for synchronizing counterpulsation therapy with the cardiac cycle.

The timing of muscular stimulation may be controlled in a number of different ways. For example, muscular stimulation may be initiated after some specified delay (e.g., 20-30 milliseconds) following detection of an R wave, where the delay is estimated to coincide with the start of mechanical diastole. Alternatively, detection of a T wave could be used as a marker to initiate muscular stimulation after a shorter delay. The start of mechanical diastole may be represented as the dichrotic notch in an aortic pressure waveform and is caused by closure of the aortic valve. The sound of aortic valve closure could also be used as a marker for the start of diastole. Once initiated, the muscular stimulation may then be delivered for a specified stimulation duration selected to lapse before the start of systole in the next cardiac cycle (e.g., 20-300 milliseconds). Alternatively, detection of an R wave or the sound of mitral valve closure may be used to terminate the stimulation. Also, multiple stimulation transducers may be disposed on an extremity and then actuated sequentially during diastole in a distal-to-proximal direction according to specified sequence parameters that specify the sequence. When multiple stimulation transducers are disposed on different body regions, more complicated stimulation sequences are also possible.

The control unit may also be configured to automatically adjust one or more stimulation parameters in closed-loop fashion based upon one or more measured physiological variables related to the patient's hemodynamics and that are affected by the counterpulsation therapy. Examples of such physiological variables include cardiac output, blood pressure, peripheral blood flow, and blood oxygen concentration. Among the stimulation parameters that may be adjusted in this manner are those that relate to the timing of the stimulation in relation to the cardiac cycle. As aforesaid, muscular stimulation may be initiated after some specified delay following detection of an R wave and then ceased after some specified stimulation duration. The specified delay and/or the specified stimulation duration in this embodiment could be automatically optimized by the control unit. In embodiments utilizing other markers of cardiac activity to initiate delivery of muscular stimulation (e.g., T waves, heart sounds), a specified delay and stimulation duration may be similarly automatically optimized. Sequence parameters used for sequential multi-transducer stimulation could also be automatically optimized. Another stimulation parameter that could be automatically optimized relates to the energy delivered to the muscular tissue. For safety and comfort reasons, it is desirable to minimize this parameter while still providing the desired therapeutic effect.

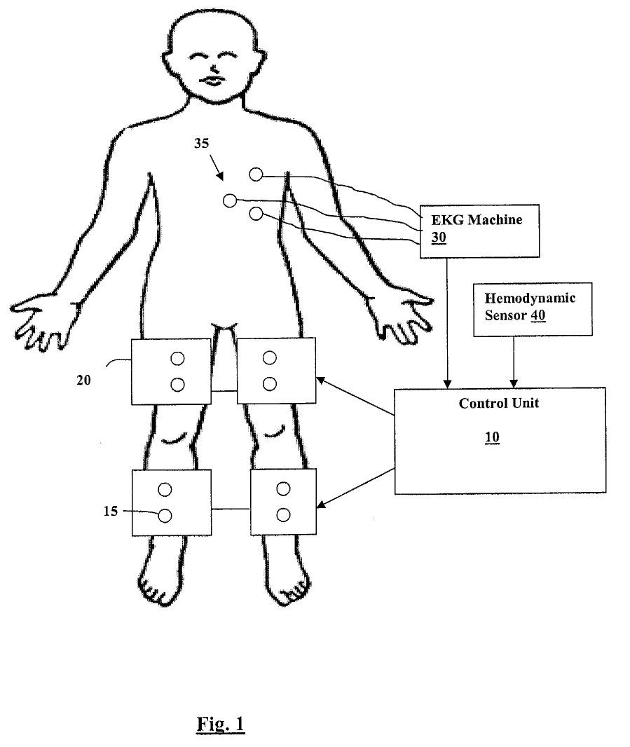

FIG. 1 illustrates one embodiment of a system for delivering counterpulsation therapy via muscular stimulation. A control unit 10 controls the delivery of counterpulsation therapy by actuating a plurality of muscle stimulation transducers 15 that may be, for example, electrodes for electrically stimulating muscular tissue. The transducers 15 in this embodiment are incorporated into a plurality of cuffs 20 that are fitted around the patient's legs so as to dispose the transducers 15 on the skin overlying selected sites. The control unit 10 in this embodiment is interfaced to an ECG apparatus 30 that includes a plurality of electrodes 35 for affixation to the patient's chest. The control unit 10 interprets signals generated by the ECG apparatus 30 to determine the phases of the cardiac cycle and deliver counterpulsation therapy in accordance therewith. A hemodynamic sensor 40 is also shown as interfaced to the control unit 10 that enables the control unit to assess the effects of the counterpulsation therapy and adjust one or more stimulation parameters accordingly. The hemodynamic sensor may be, for example, a sensor for measuring cardiac output, blood pressure, peripheral blood flow, and/or blood oxygen concentration.

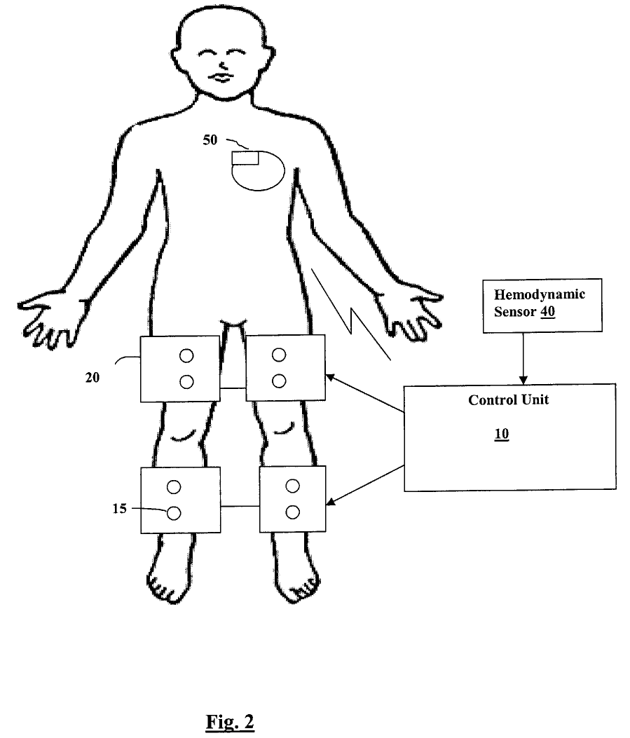

FIG. 2 illustrates an embodiment of a system for delivering counterpulsation therapy via muscular stimulation that utilizes an implantable cardiac device. This embodiment includes a control unit 10, a plurality of muscle stimulation transducers 15 incorporated into a plurality of cuffs 20 that are fitted around the patient's legs, and hemodynamic sensor 40 as described above with reference to FIG. 1. Rather than detecting cardiac activity via a surface ECG, the control unit 10 in this embodiment receives signals indicative of cardiac activity via wireless telemetry from an implantable cardiac device 50. The implantable cardiac device (e.g., a pacemaker or ICD) has sensing channels incorporating internal electrodes for detecting cardiac activity, which information is then relayed to the control unit 10. The implantable cardiac device may also incorporate other sensing modalities for measuring variables such as cardiac stroke volume or cardiac output that can be transmitted to the control unit 10. Signals from such additional sensing modalities of the implantable cardiac device may be used by the control unit 10 in addition to, or instead of, signals from an external hemodynamic sensor 40 to adjust stimulation parameters.

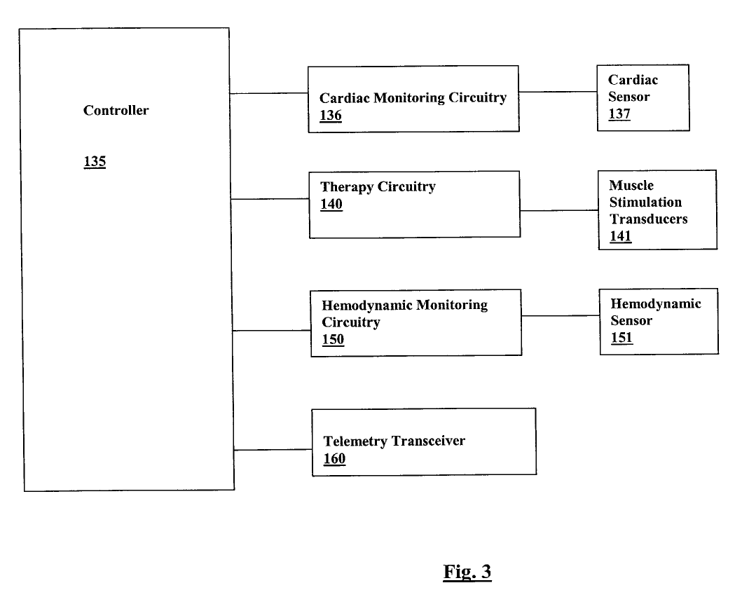

FIG. 3 illustrates the functional components of an exemplary control unit. Depending upon the particular embodiment, the control unit may include any or all of the illustrated components. A controller 135 controls the overall operation of the system. The controller 135 may be made up of discrete circuit elements but is preferably a processing element such as a microprocessor together with associated memory for program and data storage which may be programmed to perform algorithms for delivering therapy. (As the terms are used herein, “circuitry” and “controller” may refer either to a programmed processor or to dedicated hardware components configured to perform a particular task.) The controller is interfaced to cardiac monitoring circuitry 136 from which it receives data generated by one or more cardiac sensors 137. The monitoring circuitry may include, for example, circuitry for amplification, filtering, analog-to-digital conversion, and/or signal processing of voltages generated by a cardiac sensor. The controller 135 is also interfaced to therapy circuitry 140 in order to control the action of one or more muscle stimulation transducers 141 in response to conditions sensed by the cardiac monitoring circuitry 136. In the case where the muscle stimulation transducers are electrodes, the therapy circuitry 140 comprises one or more pulse generators for delivering voltage pulses to the electrodes at amplitudes determined by the controller. In the case of other types of muscle stimulation transducers, the therapy circuitry 140 comprises circuitry for energizing the transducer. The controller may then be programmed to actuate the muscle stimulation transducers during the diastolic phase of the cardiac cycle as determined by the cardiac monitoring circuitry. Also interfaced to the controller 135 is hemodynamic monitoring circuitry 150 that receives signals from one or more hemodynamic sensors 151 and enables the controller to automatically adjust stimulation parameters and optimize the counterpulsation therapy as described above. Such hemodynamic sensors may be configured, for example, to measure cardiac output, blood pressure, peripheral blood flow, and/or blood oxygen concentration. A telemetry transceiver 160 is also shown for communicating with an implantable cardiac device via wireless telemetry.

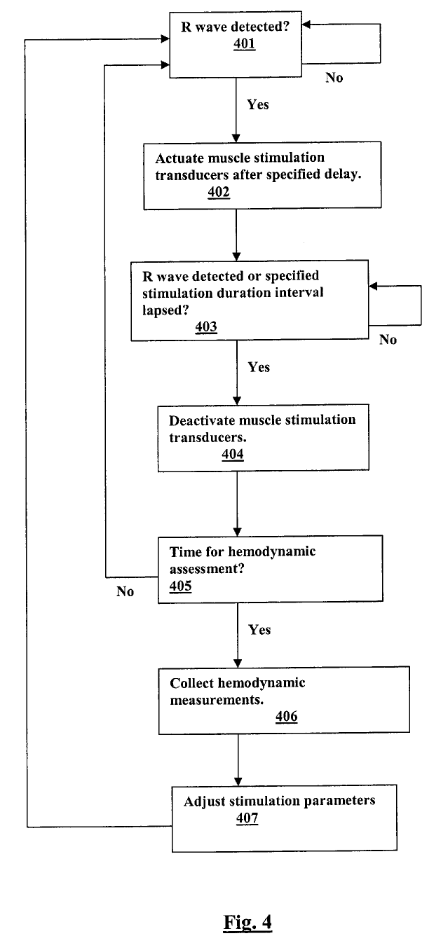

FIG. 4 illustrates an exemplary algorithm that may be executed by the controller 135 in order to deliver counterpulsation therapy. At step 401, the controller waits for detection of an R wave in an ECG or electrogram indicating the start of cardiac systole and waits for a specified delay estimated to be the beginning of diastole. At the start of diastole, the controller actuates the muscle stimulation transducers according to a specified sequence at step 402. At steps 403 and 404, the controller deactivates the muscle stimulation transducers upon detection of an R wave indicating the start of systole or upon the lapsing of a defined time interval. At step 405, the controller determines if it is time to assess the hemodynamic effects of the counterpulsation therapy as determined from measurement of one or more hemodynamic variables. The time for such assessment may be defined to occur periodically (e.g., every 15 minutes) with the hemodynamic variable measurements averaged over the defined period. If it is not time for the periodic hemodynamic assessment, the controller returns to step 401. Otherwise, the hemodynamic variable measurements are collected at step 406, and one or more stimulation parameters are adjusted in accordance therewith at step 407 before returning to step 401.

The invention has been described in conjunction with the foregoing specific embodiments. It should be appreciated that those embodiments may also be combined in any manner considered to be advantageous. Also, many alternatives, variations, and modifications will be apparent to those of ordinary skill in the art. Other such alternatives, variations, and modifications are intended to fall within the scope of the following appended claims.

What is claimed is:

1. An apparatus, comprising:
one or more muscular stimulation transducers for stimulating the skeletal and/or smooth muscle of a patient's extremity;
a cardiac sensor for detecting cardiac activity;
a control unit for actuating the one or more muscular stimulation transducers during the diastolic phase of the cardiac cycle as determined from the detected cardiac activity.

2. The apparatus of claim 1 wherein the one or more muscular stimulation transducers is a plurality of such transducers adapted for placement on a patient's extremity at proximal and distal locations and further wherein the control unit is configured to actuate the more distally located muscular stimulation transducer(s) before the more proximally located muscular stimulation transducers during diastole.

3. The apparatus of claim 1 wherein the one or more muscular stimulation transducers are of a type that is selected from a group that includes an electrode, a radio-frequency transducer, a magnetic transducer, and an ultrasonic transducer.

4. The apparatus of claim 1 wherein the one or more muscular stimulation electrodes are incorporated into a wearable structure selected from a group that includes a cuff, a sock, or pants that dispose the muscular stimulation electrodes near the muscles of an extremity.

5. The apparatus of claim 1 wherein the one or more muscular stimulation electrodes are incorporated into a patient-support structure selected from a group that includes a mat, a chair, or a recliner that dispose the muscular stimulation electrodes near the muscles of an extremity when the patient is supported thereon.

6. The apparatus of claim 1 wherein the cardiac sensor is a sensor selected from a group that includes sensors for detecting cardiac electrical activity, cardiac mechanical activity, arterial blood pressure, arterial pulse pressure, arterial/venous oxygen/carbon dioxide concentrations, and arterial blood flow.

7. The apparatus of claim 1 wherein the cardiac sensor is an external ECG monitor interfaced to the control unit.

8. The apparatus of claim 1 wherein the cardiac sensor is an implantable cardiac device interfaced to the control unit via telemetry.

9. The apparatus of claim 1 further comprising:
a sensor for measuring arterial blood flow or arterial blood pressure; and,
wherein the controller is configured to adjust the timing of actuating the muscular stimulation transducers in a manner that maximizes arterial blood flow, arterial blood pressure, are arterial blood oxygen concentration.

10. The apparatus of claim 1 further comprising:
a sensor for measuring arterial blood flow or arterial blood pressure; and,
wherein the controller is configured to adjust the stimulation energy of the muscular stimulation transducers in a manner that maximizes arterial blood flow, arterial blood pressure, or arterial blood oxygen concentration.

11. A method, comprising:
disposing one or more muscular stimulation transducers in order to stimulate the skeletal and/or smooth muscle of a patient's extremity;
detecting cardiac activity;
actuating the one or more muscular stimulation transducers during the diastolic phase of the cardiac cycle as determined from the detected cardiac activity.

12. The method of claim 11 wherein the one or more muscular stimulation transducers is a plurality of such transducers placed on a patient's extremity at proximal and distal locations and further comprising actuating the more distally located muscular stimulation transducer(s) before the more proximally located muscular stimulation transducers during diastole.

13. The method of claim 11 wherein the one or more muscular stimulation transducers are of a type that is selected from a group that includes an electrode, a radio-frequency transducer, a magnetic transducer, and an ultrasonic transducer.

14. The method of claim 11 wherein the one or more muscular stimulation electrodes are incorporated into a wearable structure selected from a group that includes a cuff, a sock, or pants that dispose the muscular stimulation electrodes near the muscles of an extremity.

15. The method of claim 1 1 wherein the one or more muscular stimulation electrodes are incorporated into a patient-support structure selected from a group that includes a mat, a chair, or a recliner that dispose the muscular stimulation electrodes near the muscles of an extremity when the patient is supported thereon.

16. The method of claim 11 wherein the cardiac sensor is a sensor selected from a group that includes sensors for detecting cardiac electrical activity, cardiac mechanical activity, arterial blood pressure, arterial pulse pressure, arterial/venous oxygen/carbon dioxide concentrations, and arterial blood flow.

17. The method of claim 11 wherein the cardiac sensor is an external ECG monitor.

18. The method of claim 11 wherein the cardiac sensor is an implantable cardiac device and further comprising receiving cardiac activity signals via telemetry.

19. The method of claim 11 further comprising:
measuring arterial blood flow or arterial blood pressure; and,
adjusting the timing of actuating the muscular stimulation transducers in a manner that maximizes arterial blood flow, arterial blood pressure, are arterial blood oxygen concentration.

20. The method of claim 11 further comprising:
measuring arterial blood flow or arterial blood pressure; and,
adjusting the stimulation energy of the muscular stimulation transducers in a manner that maximizes arterial blood flow, arterial blood pressure, or arterial blood oxygen concentration.
